(19) United States
(12) Patent Application Publication
(10) Pub. No.: US 2019/0060492 A1
(43) Pub. Date: Feb. 28, 2019

(54) SYSTEM AND METHOD OF PROVIDING DISINFECTION, DECONTAMINATION, AND STERILIZATION

(71) Applicant: DABNEY PATENTS, L.L.C., Georgetown, TX (US)

(72) Inventor: Paul Dabney, Georgetown, TX (US)

(73) Assignee: DABNEY PATENTS, L.L.C., Georgetown, TX (US)

(21) Appl. No.: 15/686,835

(22) Filed: Aug. 25, 2017

(51) Int. Cl. A61L 2/00; A61L 2/08; A61L 2/10; A61L 2/26

(52) U.S. Cl. CPC A61L 2/0076; A61L 2/0047; A61L 2/0052; A61L 2/088; A61L 2202/25; A61L 2/10; A61L 2/26; A61L 2202/11; A61L 2202/15; A61L 2/084

(57) ABSTRACT

A method, system, and apparatus for providing sterilization, decontamination, and therapeutic treatment may be provided. These may include a misting, vaporizing, spraying, foaming or other dispensing apparatus to cover a person, a surface, and enclosure, a room or other structure with an antimicrobial solution. Radiation sources, such as lights, may be utilized to expose the antimicrobial solution to a certain wavelength of radiation. The combination of the antimicrobial solution and the certain wavelength of radiation may create a synergistic reaction that causes an effect greater than the radiation or solution separately. A container or passageway including an apparatus dispensing a solution so that an object or person is saturated with an antimicrobial solution. The person or object is then exposed to a certain wavelength of radiation that creates a synergistic reaction between the solution and the radiation.

### BACKGROUND

Microbes exist that cause harm or disease in living tissues of humans and animals. Tumors, neoplasms and other manifestations of cancer also cause harm to the tissues of humans and animals. Currently a variety of manners are utilized to try to control and treat unsanitary conditions which often lead to infections, as well as methods of attempting to clean or sterilize environments or objects. However, due to the spread of infections and mutations of bacteria, like MRSA, the current methods are often ineffective or impractical. Further, environments such as doctors' offices, surgical centers and rooms, and facilities where humans interact with each other and various apparatuses, such as gyms, are often the most susceptible to creating environments that can lead to infections or other ill-effects on the patients or users of the facility. As a result, the environments where people go to receive treatment or improve their health often result in infections and other undesirable conditions.

### SUMMARY

According to an exemplary embodiment, a method, system, and apparatus for providing sterilization, decontamination, and therapeutic treatment may be provided. These may include a misting, vaporizing, spraying, foaming or other dispensing apparatus to cover a person, a surface, and enclosure, a room or other structure with an antimicrobial solution. Further, when applied to a person or animal, the antimicrobial solution may include a transdermal ingredient to aid in absorption or penetration of the skin. The antimicrobial solution may be heated. Radiation sources may be utilized to expose the antimicrobial solution to a certain wavelength of radiation. The combination of the antimicrobial solution and the certain wavelength of radiation may create a synergistic reaction that causes an effect greater than the radiation or solution separately. The synergistic reaction between the radiation and the solution may be greater than the sum of the effect of the radiation or solution applied independently. A container or passageway including an apparatus dispensing a solution may be provided so that an object, animal, or person is saturated with an antimicrobial solution. The person, animal, or object is then exposed to a certain wavelength of radiation that create a synergistic reaction between the solution and the radiation causing an effect that is greater than the effect of the solution or the radiation acting independently. Further, the radiation sources may be fixed, adjustable, or moveable.

According to another exemplary embodiment, a method of providing therapeutic treatment may be provided. The method of providing therapeutic treatment may include a misting, vaporizing, spraying, foaming or other dispensing apparatus to cover a person, an animal, a surface, an enclosure, a room or other structure with an antimicrobial solution. The antimicrobial solution may be heated to a predetermined temperature. Radiation sources may be utilized to expose the antimicrobial solution to a certain wavelength of radiation, for example light radiation. The combination of the antimicrobial solution and the certain wavelength of radiation may create a synergistic reaction that causes an effect greater than the radiation or solution separately. The synergistic reaction between the radiation and the solution may be greater than the sum of the effect of the radiation or solution applied independently to provide sterilization, sanitization, or decontamination. An apparatus dispensing a solution may be used so that an object, animal, person or room is saturated with an antimicrobial solution. The person or object or room or enclosure is then exposed to a certain wavelength of radiation from less than 1 second to over 1 minute. This creates a synergistic reaction between the solution and the radiation causing an effect that is greater than the effect of the solution or the radiation acting independently. The dispensing and radiation sources may be arranged so that complete coverage of the room, person, animal, structure, apparatus or other item or items is completely illuminated by the antimicrobial solution and radiation.

According to another exemplary embodiment, a method of providing therapeutic treatment may be provided. The method of providing therapeutic treatment may include a misting, vaporizing, spraying, foaming or other dispensing apparatus to cover a surface, an enclosure, a room or other structure with an antimicrobial solution. The surface, enclosure, room or other structure may contain tools, supplies, equipment or other devices to be exposed to the solution and certain wavelength of radiation so that the tools, supplies, equipment or other devices are rendered free of the target microbes by the synergistic reaction between the antimicrobial solution and the certain wavelength of radiation. The antimicrobial solution may be heated. Radiation sources may be utilized to expose the antimicrobial solution to a certain wavelength of radiation. The combination of the antimicrobial solution and the certain wavelength of radiation may create a synergistic reaction that causes an effect greater than the radiation or solution separately. The synergistic reaction between the radiation and the solution may be greater than the sum of the effect of the radiation or solution applied independently.

A container, duct, passageway or conduit (such as an air conditioning duct) including an apparatus dispensing a solution so that an object, area or person is saturated with an antimicrobial solution may be described in a further exemplary embodiment. The person, area, or object is then exposed to a certain wavelength of radiation that creates a synergistic reaction between the solution and the radiation, causing an effect that is greater than the effect of the solution or the radiation acting independently. This synergistic reaction enables subclinical concentrations of the anti-microbial solution and subclinical levels of radiation to provide a therapeutic effect or otherwise sterilize, disinfect, sanitize, and/or decontaminate an object, area, person, or animal.

### BRIEF DESCRIPTION OF THE DRAWINGS

Advantages of exemplary embodiments of the system, retainer and method of providing therapeutic treatment will be apparent from the following detailed description of the exemplary embodiments. The following detailed description should be considered in conjunction with the accompanying figures in which:

FIG. 1 is an exemplary diagram showing a sterilization and decontamination tower;

FIG. 2 is an exemplary diagram showing a room equipped with a decontamination and sterilization system;

FIG. 3 is an exemplary diagram showing a sterilization and decontamination chamber;

FIG. 4 is an exemplary diagram showing a sterilization and decontamination system integrated into a passageway.

FIG. 5 is an exemplary diagram showing a sanitizing station.

### DETAILED DESCRIPTION

Aspects of the present invention are disclosed in the following description and related figures directed to specific exemplary embodiments of the invention. Those skilled in the art will recognize that alternate exemplary embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The exemplary embodiments described herein are not limiting, but rather are exemplary only. It should be understood that the described exemplary embodiments are not necessarily to be construed as preferred or advantageous over other exemplary embodiments. Moreover, the terms “exemplary embodiments of the invention”, “exemplary embodiments” or “invention” do not require that all exemplary embodiments of the invention include the discussed feature, advantage or mode of operation.

In an exemplary embodiment, a system, apparatus, and method for providing decontamination and sterilization may be provided. As discussed in U.S. patent application Ser. No. 15/399,955, the contents of which are hereby incorporated in their entirety, antimicrobial solutions, when treated in certain manners, can provide therapeutic, sterilizing, and decontaminating results. For example, an antimicrobial solution or solutions may be activated or combined with certain wavelengths of radiation to eliminate or reduce microbes at a higher percentage than by the solution alone. In one embodiment, a synergistic effect between certain wavelengths of radiation and antimicrobial solutions can result in the elimination or reduction of diseases and other undesired health conditions caused by microorganisms. Exemplary embodiments described herein can provide for more effective sterilization and decontamination effects when used in accordance with the methods, systems, and apparatuses described herein.

In some exemplary embodiments, an antimicrobial solution of an H₂O₂ (hydrogen peroxide) solution, such as a liquid that may be aerosolized, with concentration of 0.3 mM or any concentration of solution that may be suitable as an antimicrobial agent. In an exemplary embodiment, hydrogen peroxide, if used alone may kill 30% of bacteria that may be exposed to it for 20 seconds. However, when irradiated with a predetermined wavelength of light, the effects of the antimicrobial agent may be significantly enhanced, as shown in the materials incorporated by reference. For example, a hydrogen peroxide solution in combination with radiation of 360 nm to 500 nm may exhibit a synergistic reaction that kills 96% or more of bacteria exposed to this combination for 20 seconds. This solution may work best at a temperature of about 57 degrees Celsius. It may be appreciated that other chemicals may have different preferred temperatures. Exemplary embodiments of other chemicals or solutions includes, but is not limited to, carbamide peroxide, benzoyl peroxide and other chemicals deemed effective may be delivered in various organic vehicles or carriers. Further, exemplary embodiments of carriers may include a combination of ethyl alcohol and propylene glycol in which the active ingredient may present in the range of from about 0.001% to about 50% by volume of the carrier. The pH of the solution may be adjusted as desired in order to provide desired results and avoid tissue irritation on people exposed to the solution. Further, the temperature of a solution may be adjusted to increase or optimize its effectiveness. In an exemplary embodiment, systemic antimicrobial agents may be used to increase the effectiveness of the treatment. The solution may be exposed to radiation in a wavelength of 360 nm to 600 nm or any other wavelength that proves effective for a certain time that may range from 1 second to greater than 1 minute.

In still other exemplary embodiments, the antimicrobial solution may be incorporated into various vehicles or carriers, including mists, along with one or more of the following ingredients: nicotinic acid or nicotinamide that may be present in concentrations from about 0.001% to 30% by volume of the carrier.

In another exemplary embodiment, the antimicrobial solution may include erythromycin base in concentrations that may present from about 0.001% to about 30% by volume of the carrier. In yet another exemplary embodiment, the antimicrobial solution may contain clindamycin phosphate methyl 7-chloro-6,7,8-trideoxy-6-(1-5methyl-trans-4-propyl-L-2pyrrolidinecarboxamido)-1-thio-L-threo-α-D-galacto-octopyranoside 2-(dihydrogen phosphate) with a concentration from about 0.001 to 30% by volume of carrier. In another exemplary embodiment, the antimicrobial solution may contain tetracycline hydrochloride in concentrations of from 0.001 to 30% by volume of the carrier; retinoids such as 6-[3-(1-adamantyl)-4-methoxy-phenyl] naphthalene-2-carboxylic acid. In still other exemplary embodiments, the antimicrobial solution carriers may include combinations of ethyl alcohol and propylene glycol, surface active agents such as lauryl ethers and lauryl esters, and other carriers effective for the desired purposes.

In further exemplary embodiments, the antimicrobial solution may be aerosolized, vaporized, converted into a mist, liquid, foam, or the like, and may be exposed to a 10 watt radiation source emitting at wavelengths from about 360 nm to about 600 nm, thus creating a synergistic effect between the solution and the radiation causing a greater reduction in microbes than with the radiation or with the solution acting alone. The radiation source may be, for example, a light, light bulb, LED, or the like, depending on application and environment.

In exemplary embodiments, and referring generally to the figures, an antimicrobial solution may be stored in one or more storage tanks. Upon activation, the antimicrobial solution may be dispensed from one or more spraying sources, such as a spray nozzle or mister, so as to cover a predetermined area or object. The dispensed antimicrobial solution may then be irradiated with a radiation source providing light radiation at predetermined wavelengths. Further, the antimicrobial solution may be implemented in a variety of devices or environments. The combination of the antimicrobial solution and the irradiation with light may then provide sterilization, sanitization, and decontamination effects on any area, object, person, or animal covered or partially covered with the antimicrobial solution and associated light irradiation.

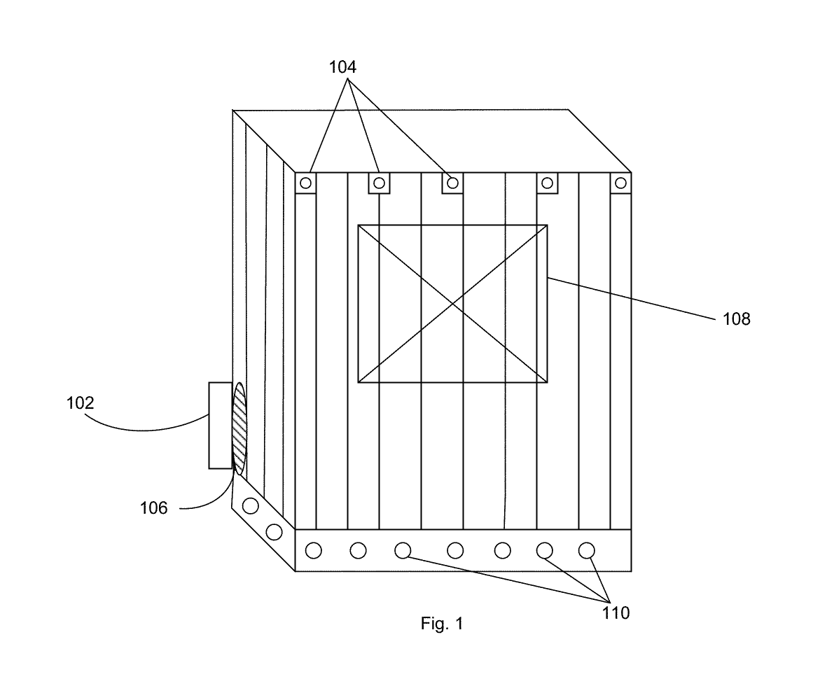

Referring now to exemplary FIG. 1, an embodiment cooling device, such as a cooling tower or mister, including a sanitization and decontamination system may be shown and described. The cooling tower 100 may be such that it is utilized for industrial, commercial, or personal usage and may be powered and formed in generally known manners for cooling devices. In use, cooling towers utilize a supply of water and the process of evaporative cooling. Water drops can be released into the air, absorbing ambient heat, and then propelled in an aerosolized or misted form by a fan or blower. However, such cooling devices are known to produce a biofilm on functional elements thereof. It is often the case that bacteria, fungus, mold, algae and other flora grow and flourish in such devices. The biofilm decreases the efficiency of the cooling device, can lead to failure of the device, and can also spread harmful or undesired flora during normal use, which can impact the health of people in the area of the cooling device.

In the exemplary embodiment of FIG. 1, cooling device 100 may have a water supply 102. Water supply 102 may be any sort of water supply, for example a conduit connected to an outside water supply that is delivered to cooling device 100 under pressure, a water storage tank, or any other form of water supply. Water supply may be fluidly connected or otherwise integrally formed antimicrobial solution storage container 106. Container 106 may contain any of the antimicrobial solutions discussed here. Further, antimicrobial storage container 106 may contain a heating element that heats or maintains the antimicrobial solution in a predetermined temperature range. Antimicrobial solution in container 106 may be mixed or intermingled with water from water supply 102 in any desired fashion. For example, an agitator may be used to mix the water and antimicrobial solution, the antimicrobial solution may be dispersed into the water supply at predetermined intervals, predetermined rates of flow of the water, or when the water is at a predetermined measured level. Additionally, water supply 102 may be heated or cooled so as to effectively provide the antimicrobial solution at a desired temperature range upon mixing.

After the water and antimicrobial solution are mixed or intermingled, the combined solution may be disseminated or otherwise moved throughout cooling device 100 in any desired fashion. Depending on the cooling device 100, the combined solution may be aerosolized after it is combined and piped to dispersing and delivery elements 104, which can include nozzles or sprayers. Alternatively, it may be maintained in liquid form and sent to dispersing and delivery elements 104 where it can be sprayed as a fine liquid or mist. A blower or air movement device 108 may then propel the combined solution in its mist or aerosolized form into the ambient environment.

Referring further to cooling device 100, one or more radiation emitting devices may be disposed on a portion of cooling device 100. The radiation emitting devices may be lights 110, and may provide light in wavelengths of about 360 nm to about 600 nm. The lights 110 may be disposed on the exterior portion of cooling device such that they provide light radiation generally on the dispersing and delivery elements 104 as well as outward where the combined solution is being sprayed. Lights 110 may be activated or illuminated in conjunction with any activation of cooling device 100. Thus, when active the combination of the light radiation and the antimicrobial solution provided by dispersing and delivery elements 104 may produce the synergistic effect described above whereby sanitization and decontamination are realized on cooling device 100 as well as in the nearby area where the antimicrobial solution is sprayed.

It may further be appreciated that the exemplary sterilization and decontamination device described above with respect to cooling device 100 may be formed in a variety of manners. For example, the device 100 could be formed as a cooling tower. Alternatively, device 100 could be formed as a fan with a mister, such as an oscillating fan. Device 100 may further be utilized in a variety of environments, for example indoor or outdoor eating areas where cooling is desired, gyms, locker rooms, athletic fields, or any other gathering area.

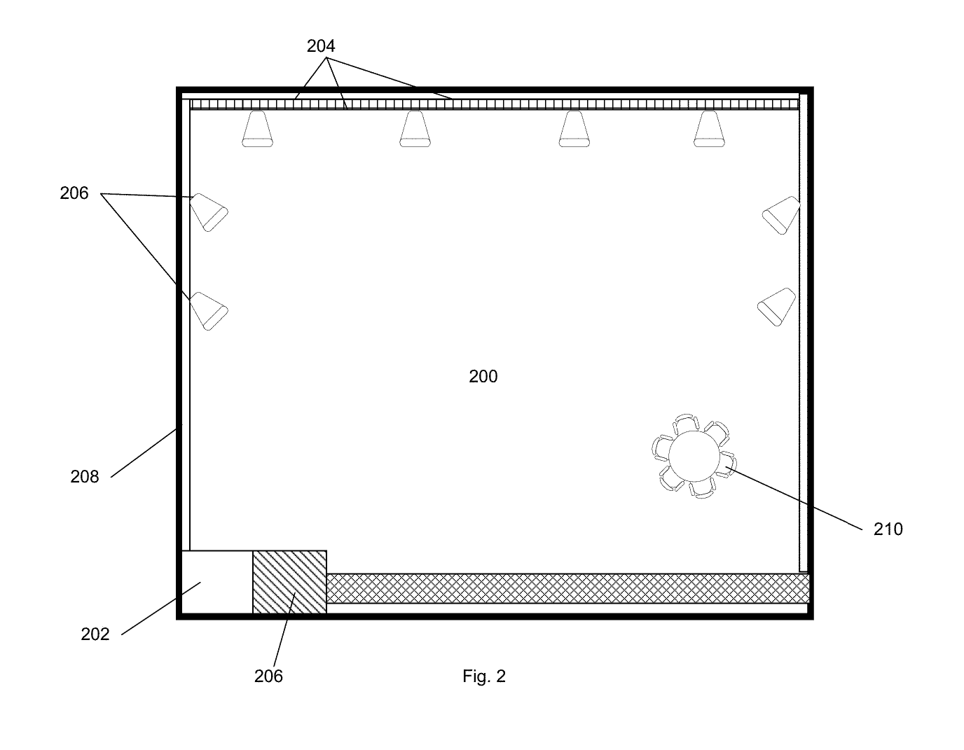

In still other exemplary embodiments, a sterilization and decontamination system may be formed in an integral or modular manner in a room. Referring now to exemplary FIG. 2, a room 200 may be shown. Room 200 may be any size or shape, as desired. Room 200 may be an operating room, doctor's office, or other such room where there are known occurrences of infection or desire to have a sterile environment, for example. Room 200 may have a variety of elements integrated or removably coupled there to, for example water supply 202 and antimicrobial solution storage container 206. As in other exemplary embodiments described herein, container 206 may be fluidly coupled with water supply 202 or directly integrated. For example, in some embodiments, room 200 may have a removable container that contains a pre-mixed solution of water and the antimicrobial solution. Room 200 may further have any numbers of conduits or pipes 208 integrated into room 200 which allow for delivery of a combined water and antimicrobial solution to misters or foggers 204. It may further be appreciated that one or more pumps (not shown) may be utilized to pump water, antimicrobial solution, or the combined water and antimicrobial solution to any desired location in room 200.

Further, in the exemplary embodiment shown in FIG. 2, after the water and antimicrobial solution are mixed to form the combined solution, the combined solution may be dispersed through any number of nozzles, foggers or misters 204 (or misting elements). Foggers or misters 204 may be nozzles that aerosolize the combined solution when it is sent through them under pressure. Alternatively, foggers or misters 204 may be any other type of fogging or misting devices, for example devices that aerosolize a liquid when it is pushed through the device under pressure. After the combined solution is sent through foggers or misters 204, a coating of the desired antimicrobial solution on any ambient features or elements of the room, such as walls, the floor, furniture, instruments, or people therein. At the same time, or at a predetermined time interval of time after the antimicrobial solution begins dispersing or is completed, one or more lights 206 may be activated to provide light in the wavelengths described above so that the synergistic effect of the combined lights and antimicrobial solution may take place, resulting in sterilization and decontamination. The lights may then remain illuminated during any misting or fogging and for a predetermined time thereafter, for example one minute or longer. In some examples, lights 206 may be activated after the misting is complete. In other exemplary embodiments, lights 206 may activate when the misting begins. In some other exemplary embodiments, lights 206 may activate at a time between the start of the misting and the completion of the misting. Lights 206 may remain on for up to, or longer than, one minute to provide the synergistic reaction with the antimicrobial solution. The lights 206 may further provide drying capabilities through heat generated in their use.

In some exemplary embodiments, room 200 may be a location where sterilization and decontamination are desirable, such as a doctor's office or operating room, as discussed above. In such embodiments, the decontamination and sterilization system could be activated at a predetermined time before a patient, doctor(s), surgeon(s), nurse(s), or other staff enter. Following activation, which may be effected by a switch or any other control, as desired, room 200 may be filled with an antimicrobial mist, fog, or vapor and lights 206 may also be activated to provide the synergistic effect. As the mist, fog, or vapor covers any items (or people) in the room, the combination of the antimicrobial solution and the desired wavelengths of light As a result, the room may be substantially sterilized and decontaminated prior to any medical procedures taking place. Additionally, or alternatively, the decontamination and sterilization system may be utilized while any of the people mentioned previously are present, after medical or surgical instruments have been introduced to the room 200, or during any medical or surgical procedure.

In further exemplary embodiment, and still referring to FIG. 2, an auxiliary lighting device 210 may be provided in room 200. The auxiliary lighting device 210 may utilize the same lights as those described with respect to lights 206 above. Additionally, lighting device 210 may be such that it is automatically activated along with lights 206. Further, lighting device 210 may be mobile or moveable, so as to provide the desired lighting in any vicinity or on any element in room 200, for example areas where lights 206 may not have a desired reach or effectiveness. Further, lighting device 210 can be utilized to provide additional light radiation on any particular element, structure, or person, as desired, in order to provide the synergistic effect of the light with the antimicrobial solution.

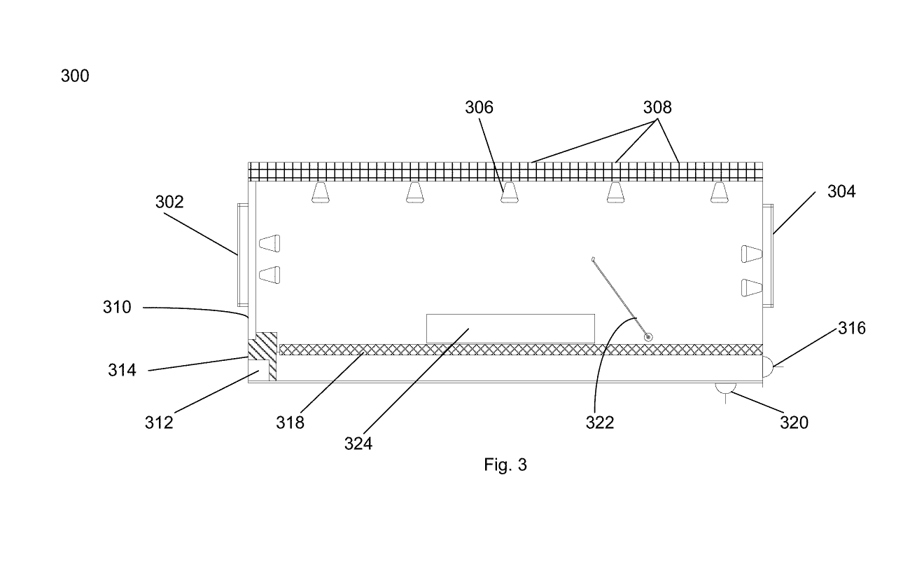

In other exemplary embodiments, a sterilization and decontamination unit can be utilized, as shown in exemplary FIG. 3. In this embodiment, sterilization and decontamination unit 300 can achieve the same synergistic effect from the antimicrobial solution and light irradiation described throughout this application. Unit 300 can be formed in any of a variety of sizes so as to accommodate different devices or to be used in different environments. For example, it may be desired to sterilize or decontaminate medical or surgical instruments before or after they are used. Alternatively, any other devices, such as cooking or eating utensils and devices, may be sterilized and decontaminated using unit 300. Thus, unit 300 may be substantially portable or other sized to accommodate commonly utilized tools or utensils. Alternatively, unit 300 could be a fixed unit that provides room and capability for the bulk sterilization and sanitization of instruments or otherwise accommodates larger items.

Unit 300 may have one or more doors or openings 302, 304 to facilitate the insertion or removal of devices from unit 300. Doors 302, 304 may be hinged, other otherwise actuatable in any desired fashion. Further, doors 302, 304 may provide a gas- and liquid-tight seal between the interior of unit 300 and the ambient environment. Further, doors 302, 304 may be locked or otherwise prevented from opening during use or when otherwise undesired.

Unit 300 may further have a top portion that includes any number of lights 306, for example lights emitting the wavelength of light that produces the synergistic effect with the antimicrobial solution, and a series of nozzles 308. Nozzles 308 may be utilized to deliver the antimicrobial solution in the form of a fog, mist, or liquid, or any other state, as desired. Antimicrobial solution may be stored in reservoir 314 and may be delivered to nozzles 308 by conduit 310, and may be propelled via pump 312. It may be noted that antimicrobial solution may be added or drained from unit 300 in any desired fashion, for example adding the solution through input 316 and draining any unused solution through drain 320. Both input 316 and drain 320 may be formed in any fashion, for example by using a removable cap or lid (not pictured).

Unit 300 may utilize any known or desired power capabilities to activate pump 312 and lights 306. Upon activation, pump 312 may pump antimicrobial solution through conduit 310 to nozzles 308. As discussed above, the antimicrobial solution may then be dispensed by the nozzles 308 in any desired form. Additionally, lights 306 may activate to provide the desired irradiation.

Unit 300 may be activated, for example, after a device or devices are placed on shelf 318. Shelf 318 may be formed in any manner and with any material, for example a stainless steel shelf formed so as to support any desired instrument, but also having any number of holes disposed there through so as to allow for drainage of any dispersed antimicrobial solution that does not evaporate. Additionally, it may be appreciated that shelf 318 may include a stand or other holding element that securely holds a device placed thereon and which may be manipulated or rotated, for example with manipulation arm 322, so as to allow a user to move the device so that is may be more thoroughly decontaminated or sterilized during use of unit 300.

Thus, a device may be inserted into unit 300 via one of doors 302, 304 and positioned on shelf 318. Door or doors 302, 304 may then be closed and sealed, and unit 300 may be activated. The antimicrobial solution may then be dispersed through nozzles 308 so that the device is substantially covered and light from lights 306 may then be provided to produce the synergistic effect caused by the combination of light at the predetermined wavelengths and the antimicrobial solution. Additionally, in some embodiments, the antimicrobial solution may be dispersed for any amount of time, as desired. Lights 306 may then be used to both achieve the synergistic effect and also effectively dry the device when the lights are left on after the completion of the dispersing of the antimicrobial solution. The device is then sanitized, sterilized and decontaminated, and removed from unit 300.

In further exemplary embodiments, unit 300 may include a timer or timing control. Depending on the size or amount of devices to be sterilized, unit 300 can be programmed or set to provide the dispersal of the antimicrobial solution for a set amount of time, provide the light irradiation for a set amount of time, and then provide further light or other methodologies to dry any devices placed therein.

In some further exemplary embodiments, tray 324 may be disposed on shelf 318. Tray 324 may be used to hold any desired bacteria samples. Then during operation, with or without a device present, the bacteria samples will be effectively killed and tray 324 can be removed to verify the destruction of the bacteria samples and effectively confirming the sterilization and decontamination of any device placed in unit 300.

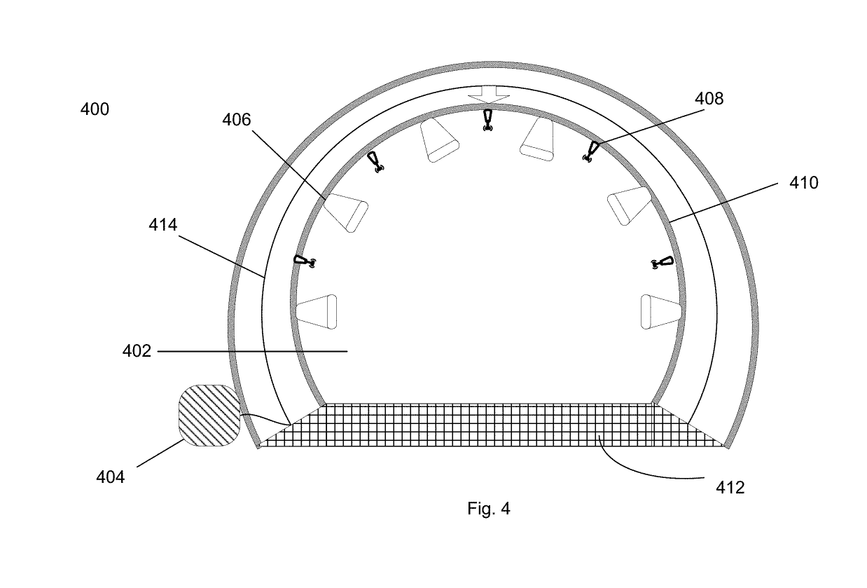

In another exemplary embodiment, and referring now to exemplary FIG. 4, a sterilization, sanitization, and decontamination system as described herein may be utilized in a doorway, passageway, or portal, or, alternatively, as a chamber. In this embodiment, sterilization and decontamination system 400 may be formed about a passageway 402. Banks of lights 406 may be formed on walls 410 and nozzles 408 may be positioned at a top portion of passageway 402, or any other desired locations in passageway 402 on walls 410. Antimicrobial solution supply 404 may be fluidly connected to one or more conduits 414 to help deliver the solution to the nozzles 408, for example with the assistance of a pump (not pictured). Antimicrobial solution supply 404 and any associated pump(s) and conduit(s) may be formed so as to be hidden or shielded from view when in use. Additionally, lights 406 and any other components utilizing electric power may be powered and/or coupled in any desired fashion. Further, nozzles 408 and lights 406 may operate in substantially the same manner as other exemplary embodiments described herein.

System 400 may allow a user to walk through or stand inside passageway 402. The system 400 can then spray the user with antimicrobial solution and use lights 406 to cause the synergistic effect which sterilizes and decontaminates the user. Further, system 400 may have a porous floor 412 which allows any excess solution or runoff to be drained from passageway 402. For example, in locations where harmful biologic agents or pathogens may be present, in addition to any other undesired microbes or bacteria, system 400 may be implemented to sterilize, decontaminate and disinfect users. In use, the system may be activated automatically, for example by an associated motion sensor, when a user walks into system 400. In different embodiments, a user may be prompted to stay in system 400 for a predetermined amount of time or may continue to walk through at a regular rate, either of which while system 400 is operating in the above-described manner. Then, at a predetermined time or after the user exists system 400, the system may deactivate. Further, it may be appreciated that system 400 can have any manual activation and deactivation controls, as desired.

In the exemplary embodiments shown herein, the antimicrobial solution and radiation of certain predetermined wavelengths described herein may be in combination with one or more of a topical antibiotic, topical anesthetic, nicotinic acid, nicotinamide, antimicrobials, salicylic acid, sulfur, retinoids such as 6[3-(1-adamantyl)-4-methoxy-phenyl] naphthalene-2-carboxylic acid, glycolic acid, tretinoin, borax, and additional chemicals useful in the method.

In other exemplary embodiments, the system and method may utilize blue light, or another certain predetermined wavelength of radiation that may supercharge the solution, with an exposure from about a few second to a few minutes. In further exemplary embodiments the antimicrobial solution may be an H₂O₂ solution, such as a gel, with concentration of 0.3 mM or any concentration of solution that may be suitable as an antimicrobial agent. In an exemplary embodiment, hydrogen peroxide, if used alone may kill 30% of bacteria that may be exposed to it for 20 seconds. Similarly, 20 seconds irradiation with radiation of the wavelength from 360 nm to 500 nm may kill 3% of bacteria that may be exposed to it. But then hydrogen peroxide in combination with radiation of 360 nm to 500 nm may exhibit a synergistic reaction that kills 96% or more of bacteria exposed to this combination for 20 seconds. This solution may work best at a temperature of about 57 degrees Celsius. It may be appreciated that other chemicals may have different functional or desired temperatures.

In an exemplary embodiment, and generally referring to FIGS. 1-5, topical solutions, including, but not limited to, hydrogen peroxide, carbamide peroxide, benzoyl peroxide and other chemicals deemed effective may be delivered in various organic vehicles or carriers. Exemplary embodiments of carriers may include a combination of ethyl alcohol and propylene glycol in which the active ingredient may present in the range of from about 0.001% to about 50% by volume of the carrier. The pH of the solution may be adjusted so that tissue sensitivity may be minimized while the effectiveness of the solution may not be hampered. The temperature of the solution may be adjusted to increase or optimize its effectiveness. In an exemplary embodiment, systemic antimicrobial agents may be used to increase the effectiveness of the treatment. The solution may be exposed to radiation in a wavelength of 360 nm to 600 nm or any other wavelength that proves effective for a certain time that may range from 1 second to greater than 1 minute.

In an additional exemplary embodiment, topical solutions of peroxide compounds may include hydrogen peroxide and/or carbamide peroxide and/or benzoyl peroxide in various organic carriers in concentrations that may range from about 0.001% to about 50% by volume of the carrier.

In another exemplary embodiment, the antimicrobial solution may contain clindamycin phosphate methyl 7-chloro-6,7,8-trideoxy-6-(1-5 methyl-trans-4-propyl-L-2pyrrolidinecarboxamido)-1-thio-L-threo-α-D-galacto-octopyranoside 2-(dihydrogen phosphate) with a concentration from about 0.001 to 30% by volume of carrier.

In another exemplary embodiment, the antimicrobial solution may contain tetracycline hydrochloride in concentrations of from 0.001 to 30% by volume of the carrier; retinoids such as 6-[3-(1-adamantyl)-4-methoxy-phenyl] naphthalene-2-carboxylic acid; It may be appreciated that the antimicrobial solution may contain any desired compounds deemed effective in treating acne vulgaris.

In still other exemplary embodiments, the antimicrobial solution carriers may include combinations of ethyl alcohol and propylene glycol, surface active agents such as lauryl ethers and lauryl esters, and other carriers effective for the desired purposes.

In other exemplary embodiments, the antimicrobial solution may include a radiation activated pigment that may fluoresce when exposed to the wavelength of radiation used in the treatment. This pigment may indicate to the user that the synergistic effect may be occurring. This synergistic antimicrobial effect may be greater than the solution's antimicrobial effect or the radiation's antimicrobial effect if they were used separately. The radiation may be applied by a light exposing a surface's entire infected area or a portion of the infected area at one time. It may be exposed to a 10 watt radiation source emitting at wavelengths from about 400 nm to about 500 nm thus creating a synergistic effect between the solution and the radiation causing a greater reduction in microbes than with the radiation or with the solution acting alone. The retainer may have a number of terminations. This particular size would enable targeting of various size areas. However, the diameter of the termination may have any desired value. The exposure time of the radiation may be about one minute. However, it may be appreciated that any desired duration of treatment may be selected.

In another exemplary embodiment, a solution containing chemical species including, but not limited to, about 15% carbamide peroxide, about 2.5% clindamycin phosphate (Methyl 7-chloro-6,7,8-trideoxy-6-(1-methyl-trans-4-propyl-L-2-pyrrolidinecarboxamido)-1thio-L-threo-α-D-galacto-octopyranoside 2-(dihydrogen phosphate)) and tretinoin may be combined in a gel-form carrier. The infected area may be exposed to radiation of a wavelength from about 410 nm to about 500 nm by a number of radiation terminations that would expose an area with a diameter of about 30 centimeters. The infected area and solution may be exposed to this certain wavelength of radiation for about 30 seconds. The synergistic effect of the radiation and solution that may be warmed may be greater than the effect of the radiation or the solution acting individually.

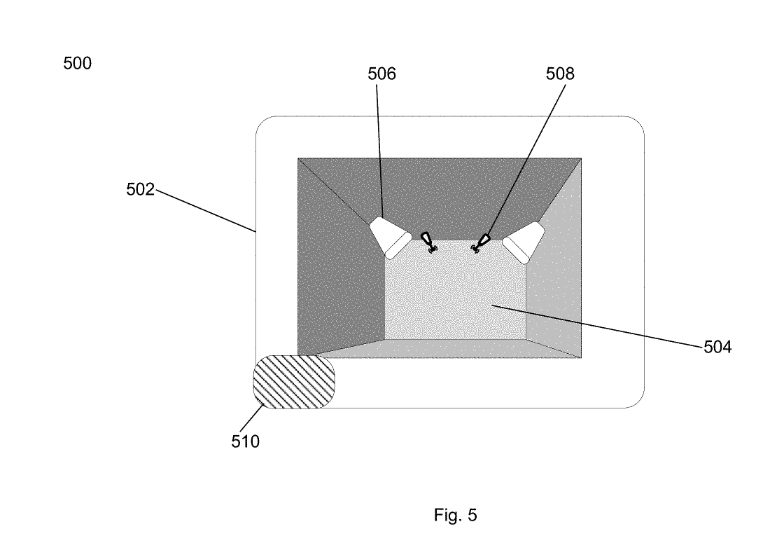

Turning now to exemplary FIG. 5, a sanitization station may be shown and described. The sanitization station 500 may be formed. Sanitization station 500 may be utilized in a similar fashion to other exemplary embodiments described herein, such as unit 300 in exemplary FIG. 3. However, station 500 may have a body 502 and an open area 504. Open area 504 may include any number of lights 506 and nozzles 508. Further, station 500 may be powered in any known manner and activated in any desired manner, for example motion detectors or switch. Thus, upon insertion of a user's hands, or the insertion of a tool or device into open area 502, station 500 may activate. Nozzles 508 may disperse an antimicrobial solution, stored in tank 510, into open area 502, covering anything placed therein. Further, lights 506 may then be activated and a user may be prompted to keep their hands (or the device) in open area 502 for a predetermined amount of time. As a result, station 500 may provide sanitization and the other beneficial, synergistic effects of combining the antimicrobial solution with the predetermined wavelengths of light.

Thus, in use, sanitization station 500 may allow for the synergistic effect of the combined antimicrobial solution and light radiation to quickly and easily sanitize and sterilize a person's hands, gloved hands, instruments, or the like. Station 500 can be deployed in any desired environment, for example in an operating room, and may be utilized at any desired time, for example after a surgeon or nurse has scrubbed in. The open area 502 can allow for the desired effects to be quickly and efficiently realized without any other contact with devices, tools, or instruments that may not be sanitized or sterilized.

The foregoing description and accompanying figures illustrate the principles, preferred exemplary embodiments and modes of operation of the invention. However, the invention should not be construed as being limited to the particular exemplary embodiments discussed above. Additional variations of the exemplary embodiments discussed above will be appreciated by those skilled in the art.

Therefore, the above-described exemplary embodiments should be regarded as illustrative rather than restrictive. Accordingly, it should be appreciated that variations to those exemplary embodiments can be made by those skilled in the art without departing from the scope of the invention as defined by the following claims.

What is claimed is:

1. A sanitization system, comprising:
a sanitization area comprising:
an open area defined in the structure;
a power source;
a tank containing an antimicrobial solution;
at least one conduit;
one or more dispersing elements fluidly coupled with the conduit, where the antimicrobial solution is delivered to the one or more dispersing elements via the conduit;
at least one radiation device; and
an open area in the structure;
wherein, upon activation, the antimicrobial solution is sent from the reservoir to the one or more dispersing elements and the antimicrobial solution is dispersed into the open area; and at a predetermined time after the antimicrobial solution is dispersed into the area, the at least one radiation device is activated.

2. The sanitization system of claim 1, further comprising a pump that facilitates the delivery of the antimicrobial solution to the one or more dispersing elements.

3. The sanitization system of claim 1, wherein the antimicrobial solution is dispersed as at least one of a vapor or liquid.

4. The sanitization system of claim 1, further comprising a floor having a plurality of openings.

5. The sanitization system of claim 1, further comprising a collection reservoir located beneath the floor that collects excess antimicrobial solution in liquid form.

6. The sanitization system of claim 1, wherein the at least one radiation source is a light that emits light in a wavelength of 360 nm to 500 nm for a predetermined amount of time.

7. The sanitization system of claim 6, wherein the light is emitted after the completion of the dispersal of the antimicrobial solution.

8. The sanitization system of claim 1, wherein the antimicrobial solution comprises hydrogen peroxide.

9. The sanitization system of claim 8, wherein the antimicrobial solution is diluted with water from a water supply.

10. The sanitization system of claim 1, wherein the antimicrobial solution is dispersed for at least ten seconds.

11. The sanitization system of claim 1, wherein the radiation device provides radiation for at least 20 seconds.

12. The sanitization system of claim 1, further comprising a heating element that heats the antimicrobial solution to a predetermined temperature range.

13. The sanitization system of claim 1, wherein the sanitization area is a passageway.

14. The sanitization system of claim 1, wherein the sanitization area is an operating room.

15. The sanitization system of claim 1, further comprising one or more portable radiation devices.

16. The sanitization system of claim 1, wherein dispersed antimicrobial solution, in combination with radiation from the at least one radiation device, provides a sanitization effect to an object or user in the sanitization system.
